(19) United States
(12) Patent Application Publication
(10) Pub. No.: US 2014/0012092 A1
(43) Pub. Date: Jan. 9, 2014

(54) DEVICE FOR MEDICAL TESTING, KIT, AND RELATED METHODS

(71) Applicant: MobileMedTek Holdings, Inc., CRESTWOOD, KY (US)

(72) Inventors: Bryan Dale Ehret, Crestwood, KY (US); Robert Trent Bashore, Pewee Valley, KY (US)

(21) Appl. No.: 13/936,470

(22) Filed: Jul. 8, 2013

Related U.S. Application Data
(60) Provisional application No. 61/668,792, filed on Jul. 6, 2012.

(51) Int. Cl. A61B 5/04; A61B 5/00

(52) U.S. Cl. CPC A61B 5/04; A61B 5/742
USPC 600/300

(57) ABSTRACT

A system is provided which will allow a medical professional to perform electrophysiological testing in a variety of locations without the inconvenience or risk involved with transporting expensive, bulky devices. This system allows the medical professional to perform a multitude of electrophysiological tests with one small, portable, affordable device, and also allow the results to be viewed remotely, if desired. The accompanying connectors, such as wiring, are adapted for use with such a device in an easy and efficient manner. This system also allows for the results of the electrophysiological tests available to medical professionals in a multitude of locations and in real time.

This application claims the benefit of U.S. Provisional Patent Application Ser. No. 61/668,792, the disclosure of which is incorporated herein by reference.

### FIELD OF THE INVENTION

This disclosure is directed to medical testing and, more particularly, to a device that facilitates the performance of one or more electrophysiological tests.

### BACKGROUND OF THE INVENTION

A multitude of electrophysiological tests are currently available for medical professionals to perform on subjects in order to analyze the electrical processes of the body, including, but not limited to, electroencephalography (EEG), electromyography (EMG), nerve conduction studies (NCS), evoked potentials (EP), transcutaneous electrical nerve stimulation (TENS), Holter monitoring, and electrocardiography (ECG/EKG). Such electrophysiological tests are currently performed via a multitude of expensive, bulky, limited mobility devices. This limited mobility hinders the medical professional's ability to perform such electrophysiological tests in a multitude of locations and on a variety of subjects, including, but not limited to, non-ambulatory subjects. Medical professionals must either own or otherwise have access to a variety of testing devices, all of which must be maintained. In addition, there are a variety of tests which could provide more comprehensive results if performed over a length of time or at a location that is infeasible for the medical professional to accommodate due to limited availability of exam room space or the physical presence of the medical professional. For example, a subject who may be experiencing intermittent heart palpitations may not manifest said palpitations during a standard test performed in the office of a medical professional, and a lengthy, portable, recorded test would offer higher testing yield.

A system must be provided which will allow a medical professional to perform electrophysiological testing in a variety of locations without the inconvenience or risk involved with transporting expensive, bulky devices. This system should allow the medical professional to perform a multitude of electrophysiological tests with one small, portable, affordable device, and also allow the results to be viewed remotely, if desired. The accompanying connectors, such as wiring, would also be adapted for use with such a device in an easy and efficient manner. This system should also make the results of the electrophysiological tests available to medical professionals in a multitude of locations and in real time.

### SUMMARY OF THE INVENTION

One aspect of the disclosure pertains to an apparatus, comprising a body including a base and a lid having an open condition and a closed condition relative to the base, and a display for holding the lid in the open condition relative to the base. The display (such as a tablet computer) may be connected to the lid, and may be releasably attached to the base. The lid may include a recess for receiving the display, and the base may include a retainer for mating with a portion of the display. The display may further include a removable sleeve including the portion for mating with the retainer. The apparatus may further include a closure for securing the lid to the base in the closed condition.

The apparatus may be adapted for use in connection with medical testing and, in particular, performing a physiological test. In such case, the base may include a connector for connecting with at least one of a plurality of electrophysiological wires.

Another aspect of the disclosure relates to an apparatus for use in performing an electrophysiological test. The apparatus comprises a bundle comprising a plurality of wires adapted for use in performing the physiological test, said plurality of wires being removably attached to each other, such as by an adhesive. The apparatus may further include a retainer for retaining the wires in the bundle, which may take the form of a ring adapted to move along the bundle in a longitudinal direction. The apparatus may further include a plug at one end of the bundle for interconnecting the plurality of wires.

A further aspect of the disclosure pertains to an apparatus for use in performing an electrophysiological test. The apparatus comprises an electrode including a recess. A substance is removably positioned in the recess. A removable cover is provided for covering the conductive substance in the recess. A wire may be provided for connecting to the electrode, which wire may comprise a first portion detachably connected to a second portion including the electrode. The first portion may include a connector for connecting with a device for receiving output signals from the wire in the course of the electrophysiological test.

A further aspect of the disclosure relates to an electrophysiological testing wire comprising a first segment removably attached to a second segment including an electrode. A connector may be provided for removably connecting the first and second segments. Preferably, the second segment is for single-use only.

Still a further aspect of the disclosure, a method for performing a medical test comprises performing the medical test at a patient location, displaying the results of the test at the patient location, and transmitting the results of the test to a remote location. The displaying and transmitting steps occur simultaneously. The transmitting step may comprise providing an input to a display device for performing the displaying step.

### BRIEF DESCRIPTION OF THE DRAWINGS

FIG. 1 is a perspective view of a device forming one aspect of the disclosure;

FIG. 2 is a front view of the device of FIG. 1;

FIG. 3 is a side view of the device of FIG. 1;

FIG. 4 is another perspective view of the device of FIG. 1;

FIG. 5 is a partially cutaway, expanded front view of the device of FIG. 1;

FIG. 6 is a partially cutaway front view of the device of FIG. 1;

FIG. 7 is a schematic view of a possible environment of use of the device of FIG. 1;

FIG. 8 is a device forming a further aspect of the disclosure;

FIG. 9 is an end view of the device of FIG. 8;

FIG. 10 is a view of a device forming a further aspect of this disclosure;

FIG. 11 is a cross-sectional view of the device of FIG. 11; and

FIG. 12 is a schematic view of a device forming a further aspect of this disclosure.

### DETAILED DESCRIPTION OF THE INVENTION

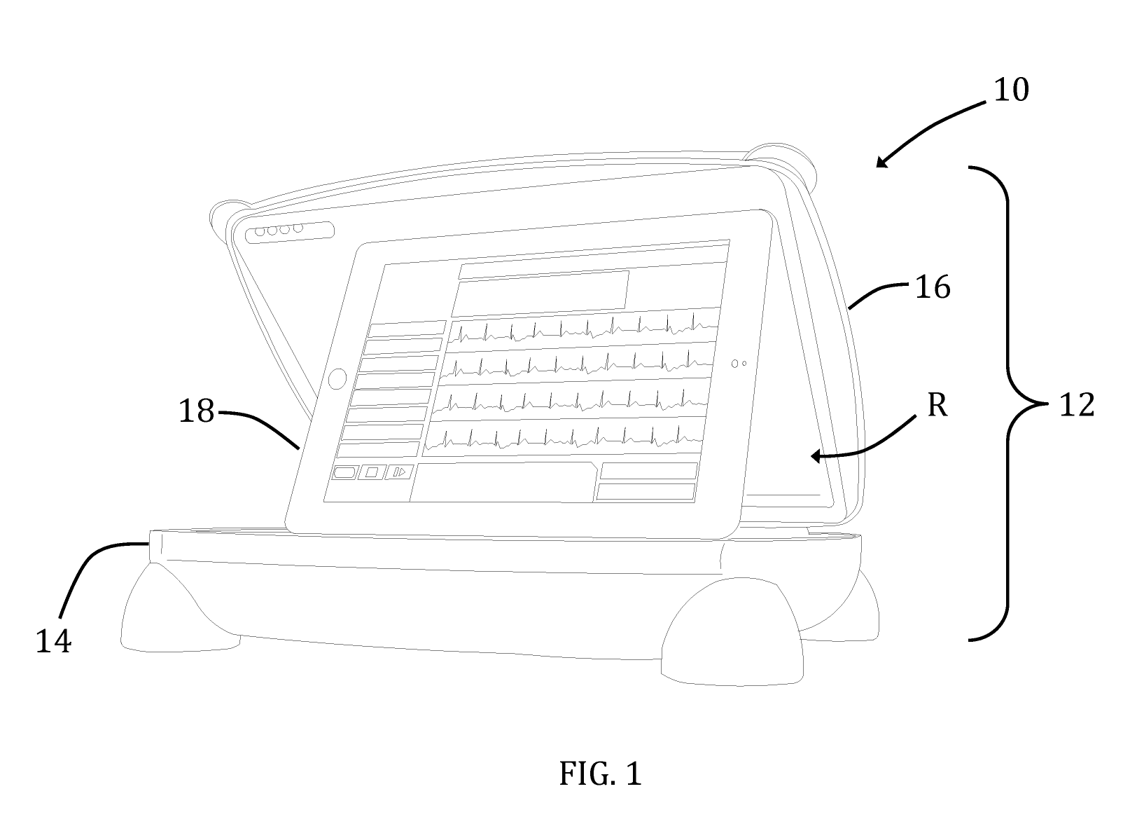

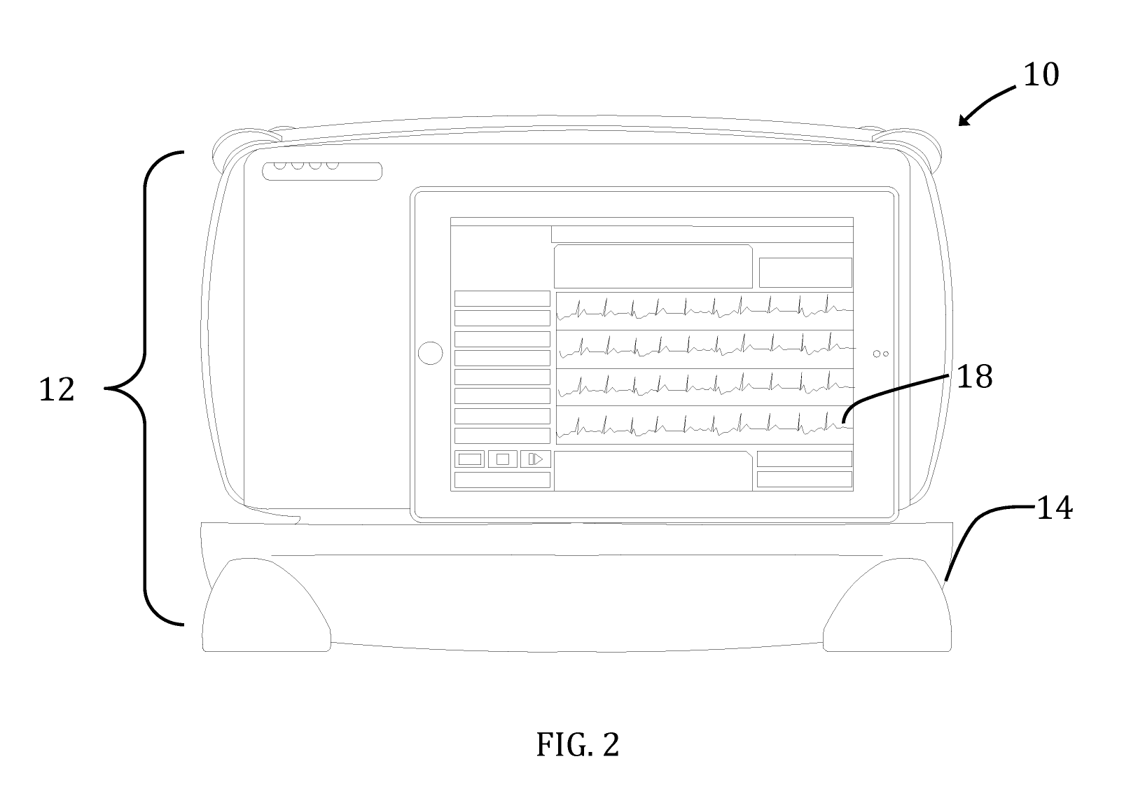

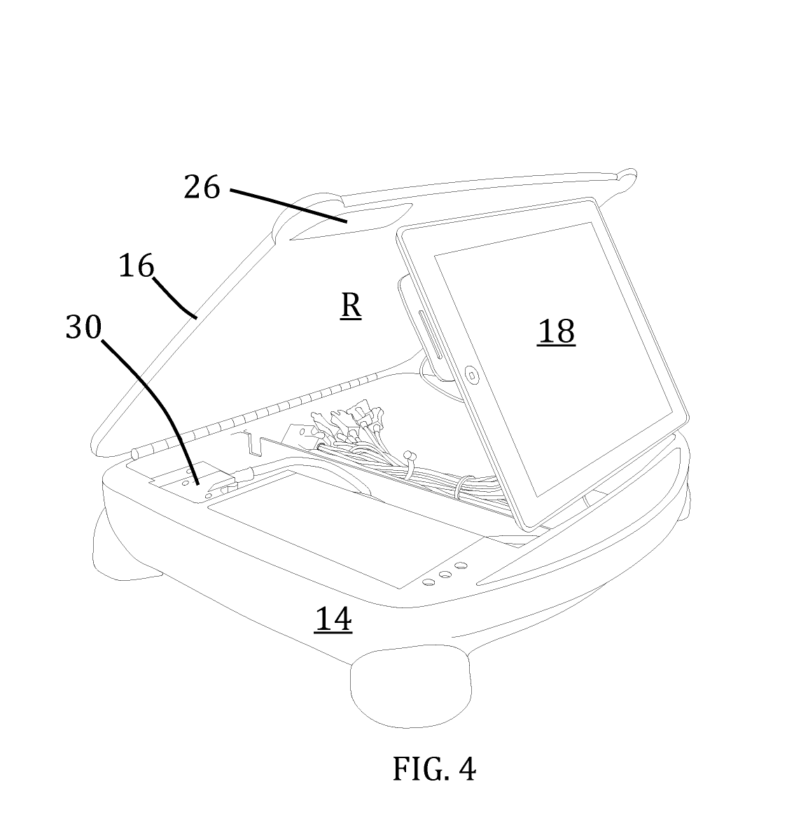

FIGS. 1-5 illustrate one embodiment of a portable device 10 that may be used in performing medical testing, such as is described in U.S. patent application Ser. No. 13/038,940, the disclosure of which is incorporated herein by reference.

In the illustrated embodiment, the device 10 comprises a case 12 including a base 14 connected to a lid 16. The connection between the base 14 and lid 16 may allow the lid 16 to move relative to the base 14 between an open condition, as shown, and a closed condition (for which a closure may be provided). This may be accomplished, for example, by using a hinged connection of any known type, but is it also possible to make the connection such that the lid 16 simply detaches from the base 14.

In the open condition, a display 18 that is normally retained within the case 12 when closed may be used to retain the lid 16 in the open condition. Specifically, the display 18, which may comprise a tablet computer (such as for example an IPAD device), may be hingedly connected to the case 12, such as along the lid 16. Consequently, the display 18 may thus pivot from a recessed condition in the case 12 (and, in particular, from a recess R formed in the lid 16) to a position for holding the lid 16 in the open condition, with the display 18 remaining viewable to a user (that is, the display 18 faces opposite the lid 16 and the base 14).

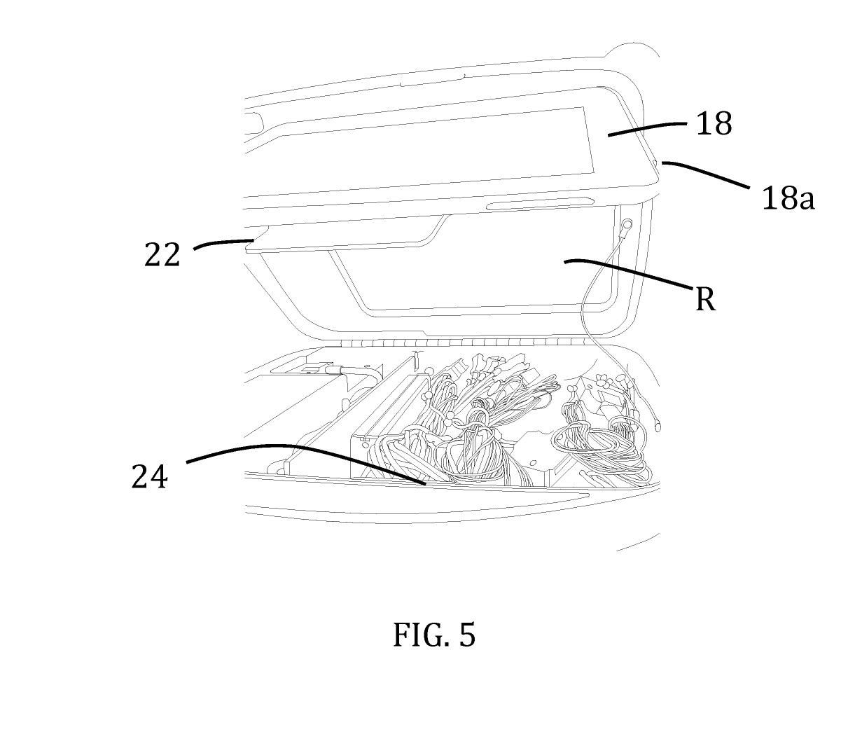

The free end 18a of the display 18 may be arranged to engage the base 14 in order to assume the position desired for holding the lid 16 in the open condition. Alternatively, as shown in FIG. 5, a projection 22 may be provided on the display 18 for engaging a retainer, such as a slot 24, on the base 14. The projection 22 may be arranged to snap-fit in this slot 24 in order to ensure that a secure, but removable, connection is established. To facilitate adding the projection 22 to the display 18, it may be associated with a sleeve 18a for receiving the display 18, which sleeve may also form the connection with the lid 16 of the case 12.

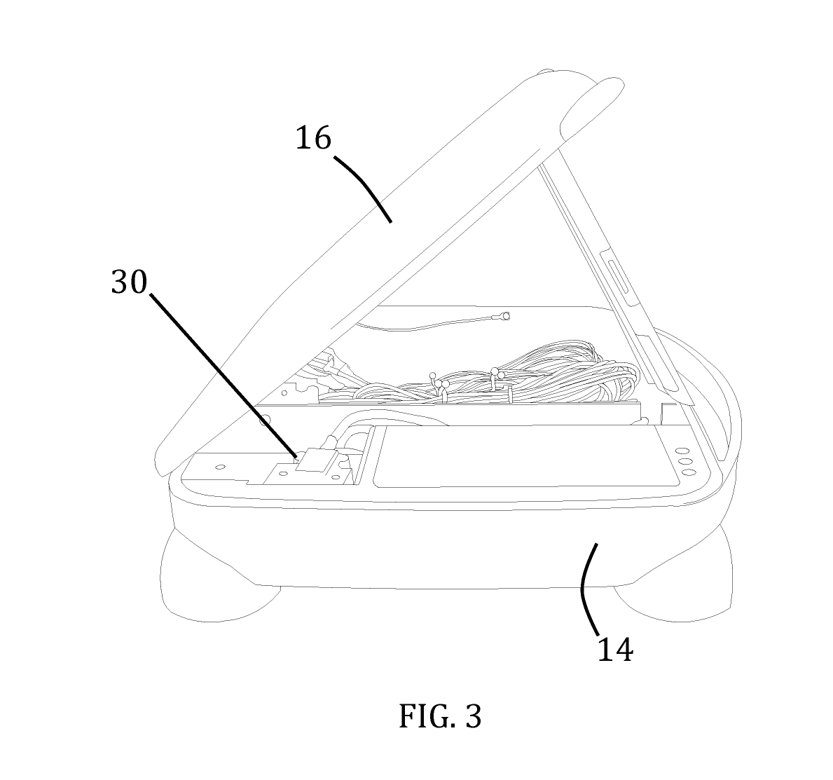

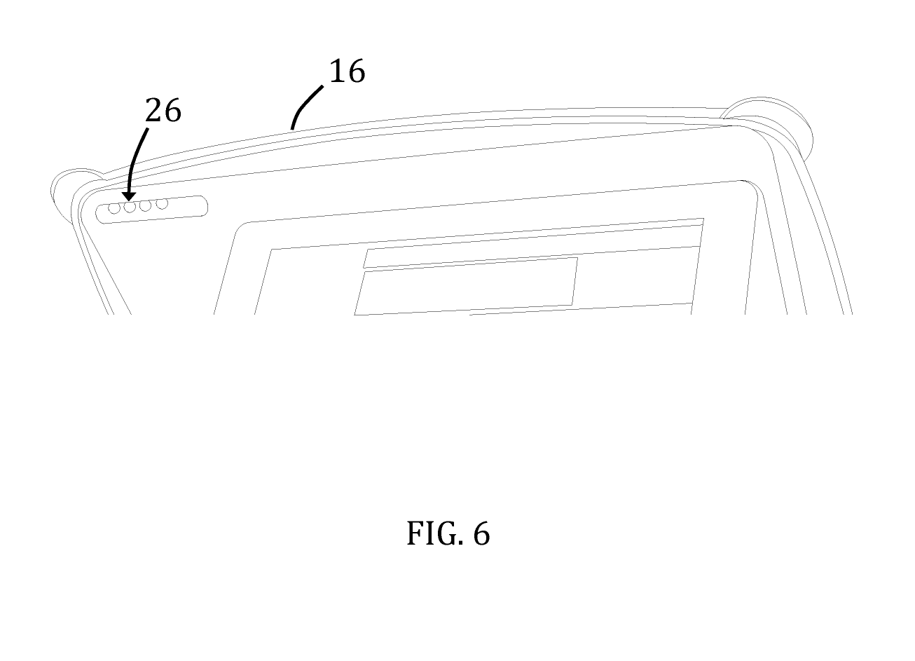

Turning back to FIG. 3, it can be seen that the device 10 may be adapted to connect with one or more wires used for performing a medical test, which may include a connector 30 (which may be secured to the lid 16, base 14, or both). The output signals from these wires may be delivered through circuitry in the device 10, such as in the base 14 or lid 16, and may be processed for display on display 18 (either by direct connection via wires, or wirelessly). With reference to FIG. 6, the device may also include an indicator, such as one or more lights 26, which serves to indicate that a signal is being received through the wires via the connection to the device 10. In the illustrated example, a first light is shown as being green in color to indicate the receipt of the signals, and lights that are different colors (yellow, red, etc.) to indicate that the connection is not established (such as if a connector associated with a wire is not present) or the connection is established, but has been interrupted (such as by a lack of a physical connection to the subject undergoing to medical testing).

Turning to FIG. 1, it can be understood that the display 18 may serve to display one or more aspects of the medical test being performed, such as in a graphical format. The device will normally be present at the site of the testing and connected directly to the subject, but it should be appreciated that a professional for observing and interpreting the signals being displayed may be located at a remote location. To facilitate the review by this person, the signals may be transmitted remotely to the location of the professional, such as by wireless transmission.

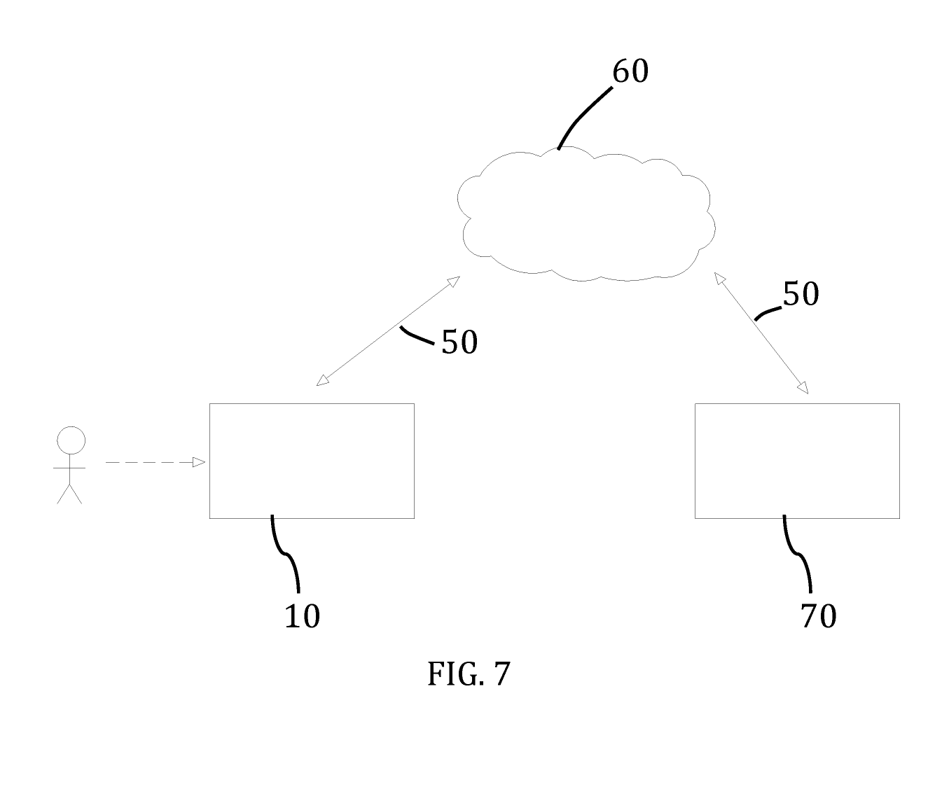

In one particular embodiment, and with reference to FIG. 7, the transmission is achieved by uploading the data representative of the signals to a network 50, such as the Internet, and then transmitting the signals to the remote location. It is also possible to upload the signals to a virtual location, such as a “cloud” 60, in order to be accessed by the professional on a device 70 at a remote location, either in real time or after the testing is completed. The transmission of the signals may be effected by the use of a controller associated with the device 10, which may be activated using the display 18 (which may have a touch screen input). The connection may also allow for communication between the patient location and the remote location, such as by audio or visual means.

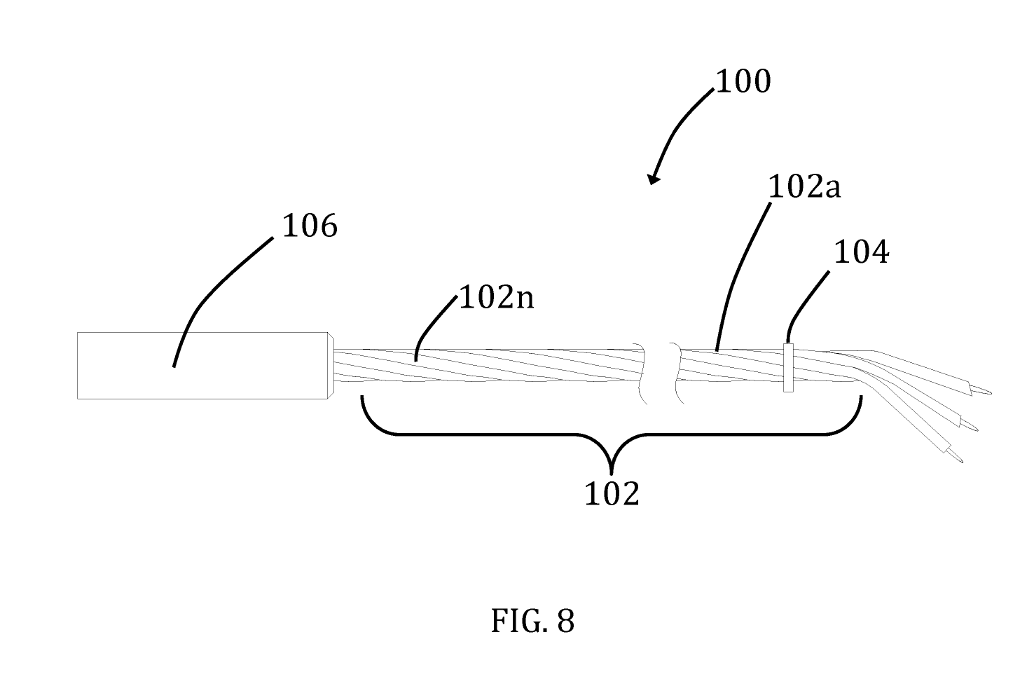

Another aspect of this disclosure pertains to particular arrangements of connections for use in performing testing, whether with the device 10 or otherwise. In one embodiment, as shown in FIG. 8, the connector 100 may comprise a bundle 102 including a plurality of wires 102a . . . 102n for transmitting signals from one end, such as from an electrode or pad connected to the body, to another end, such as a jack for connecting to an apparatus for processing the signals, such as device 10. As can be appreciated, when multiple wires are present, they can quickly become tangled and unwieldy, which leads to increased time and effort to perform the test.

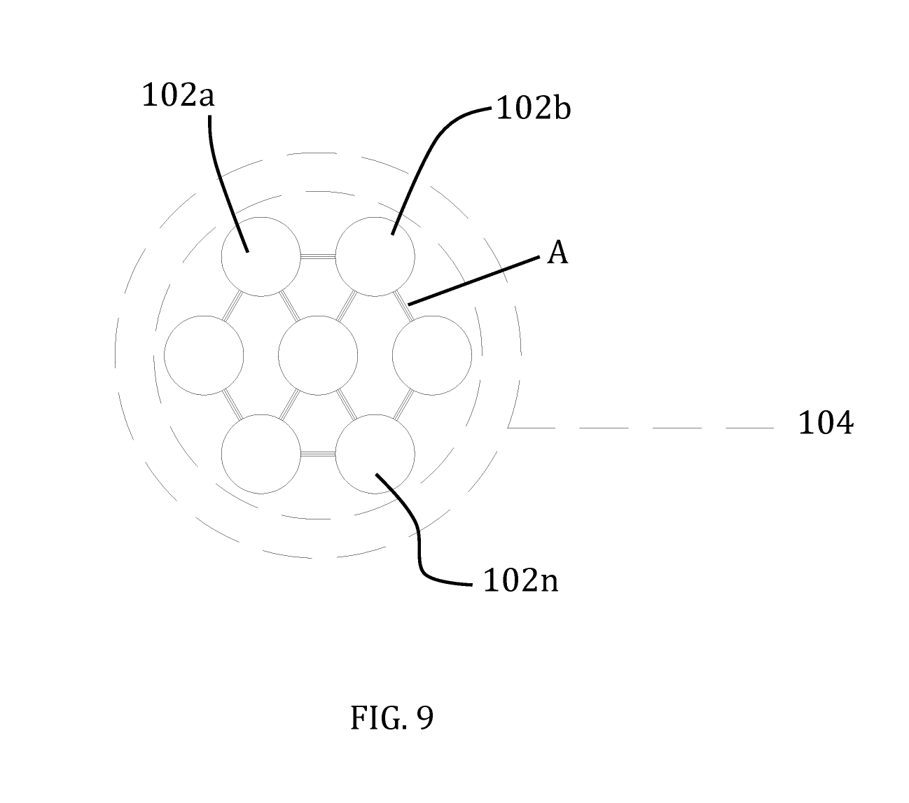

To help prevent this, and with reference to FIG. 9, at least two of the individual wires, such as wires 102a, 102b may be initially connected using an adhesive A or other form of releasable connection (such as by forming a frangible bond between two or more of the individual wires that may be separated prior to use; spacing in FIG. 9 is exaggerated to illustrate the bond formation). The connection may be established between each individual wire and all adjacent wires that contact it (which may be one or more wires), and may extend in the longitudinal direction along a majority of the wires (but may be continuous or intermittent). The individual wires 102a . . . 102n may then be peeled apart from the bundle 102 prior to or during use, such as in the course of performing a medical test involving the wires. The adhesive should be sufficiently tacky to hold the wires together when proximate each other, but releasable with the application of a relatively light manual pulling force. The adhesive may also be capable of repeated use in terms of bonding the adjacent wires 102a . . . 102n together.

The wire bundle 102 may also be associated with a retainer. The retainer may take the form of a flexible ring or band 104. This band 104 may be moved along the wires 102a . . . 102n of the bundle, such as by sliding, rolling, or a combination of the two. This may be done to bring the wires together up to a point along their longitudinal axis, and help reform the bundled condition (including when the testing operation is complete). Another form of retainer may also be provided in the nature of a plug 106 for associating the wires with a particular connector on a testing apparatus, such as device 10.

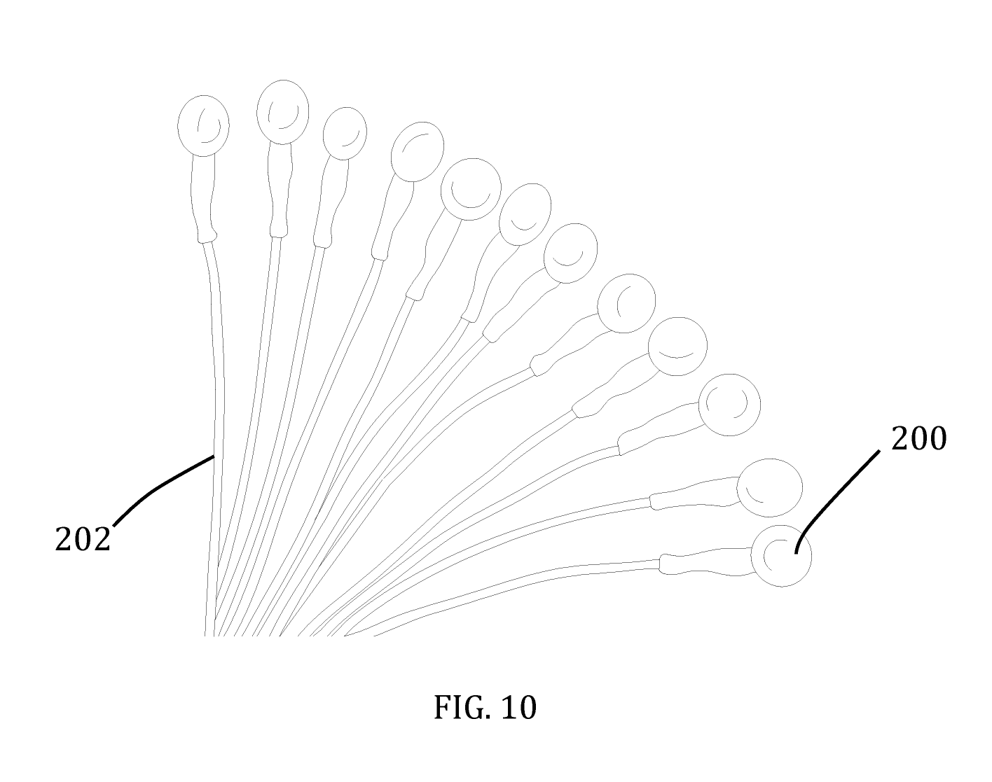

Referring to FIG. 10, another aspect of the invention relates to the electrodes 200 used in connection with the wires 202 for performing certain tests and, in particular, those that require the use of a viscous liquid substance (typically collodion in gel or paste form) in order to help form an electrical connection with the particular portion of the body involved in the testing. Usually, this substance is applied to the body and then the electrodes (which as shown may be disc-shaped) connected to it, which is a relatively messy and involved undertaking.

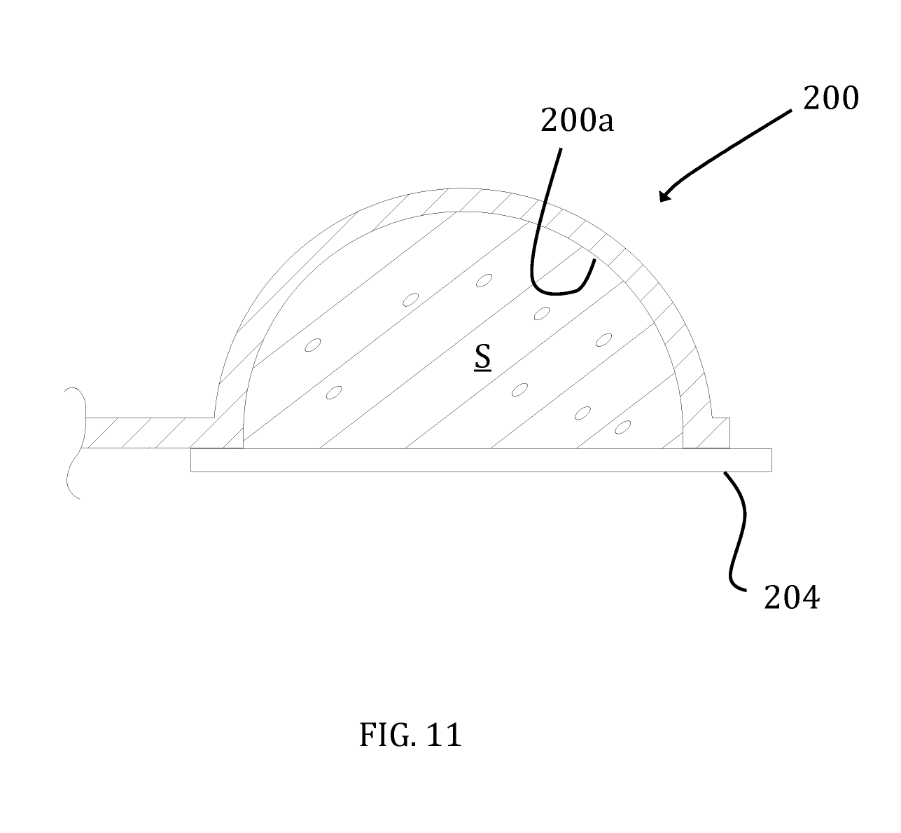

To avoid this, and with reference to FIG. 11, the substance may be pre-disposed in the electrode 200 prior to use, such as within a cavity 200a. The cavity 200a including the substance S may also be covered by a removable cover 204, such as a thin flexible substrate, that helps retain the substance in place prior to use. The cover 204 may be attached to the electrode 200, such as by an adhesive.

When use of the electrode 200 is desired, the cover 204 may simply be removed, and the electrode placed at the desired location for performing the test. As should be appreciated, this greatly enhances the efficiency of the process, including by eliminating the need to provide a separate source of the substance, and reducing the clean-up time once the testing is completed. The electrodes 200 may also be recycled for use, such as by being sterilized, refilled, and recovered.

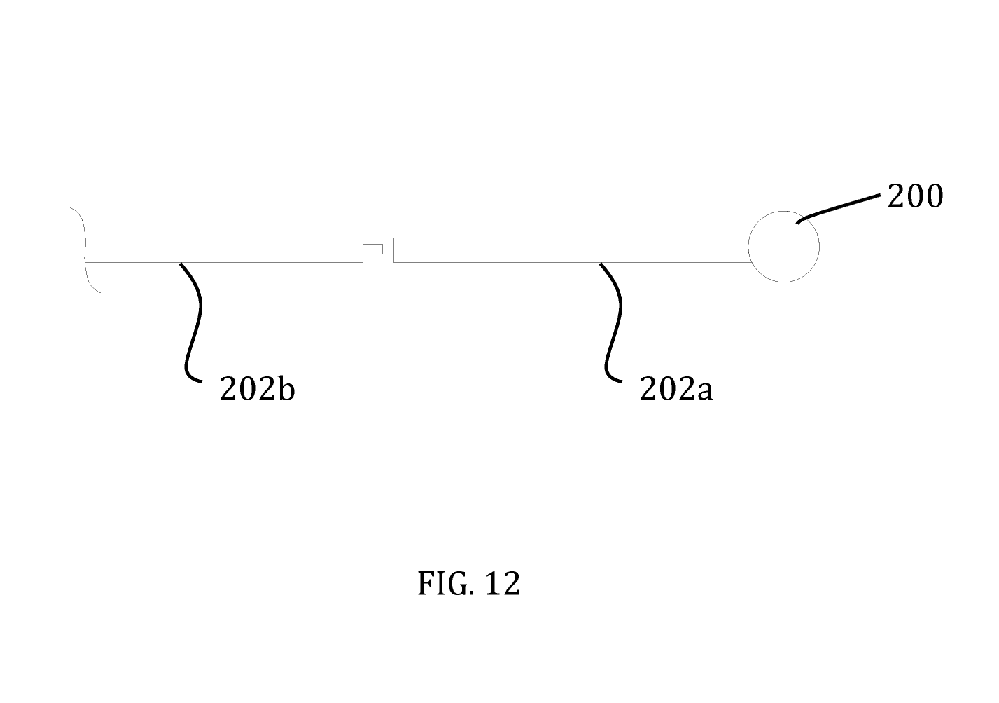

Additionally or alternatively, each wire 202 may be segmented, as shown in FIG. 12, such that the electrode 200 and the associated lead 202a may be removed from a corresponding segment 202b of the complete wire (including the connector for connecting to a device for receiving and interpreting the output signals, such as device 10). The electrode 200 and lead 202a may then be disposed of after a single use and replaced with a new one, or else cleaned and reused. The connection may be made by any suitable arrangement (such as a male plug and a female receptacle) that is capable of transmitting the signals during the performance of the electrophysiological test.

The foregoing descriptions of several embodiments made according to the disclosure of certain inventive principles herein are presented for purposes of illustration and description. The embodiments described are not intended to be exhaustive or to limit the invention to the precise form disclosed and, in fact, any combination of the components of the disclosed embodiments is contemplated. Modifications or variations are possible in light of the above teachings. The embodiments described were chosen to provide the best illustration of the principles of the invention and its practical application to thereby enable one of ordinary skill in the art to utilize the invention in various embodiments and with various modifications as are suited to the particular use contemplated. All such modifications and variations are within the scope of the invention when interpreted in accordance with the breadth to which it is fairly, legally, and equitably entitled. The disclosure of U.S. Pat. No. 8,255,238 is incorporated herein by reference.

1. An apparatus, comprising:
a body including a base and a lid having an open condition and a closed condition relative to the base; and
a display for holding the lid in the open condition relative to the base.

2. The apparatus of claim 1, wherein the display is connected to the lid.

3. The apparatus of claim 1, wherein the display is releasably attached to the base.

4. The apparatus of claim 1, wherein the lid includes a recess for receiving the display.

5. The apparatus of claim 1, wherein the base includes a retainer for mating with a portion of the display.

6. The apparatus of claim 5, wherein the display includes a removable sleeve including the portion for mating with the retainer.

7. The apparatus of claim 1, wherein the display comprises a tablet computer.

8. The apparatus of claim 1, wherein the base includes a connector for connecting with at least one of a plurality of electrophysiological wires. base.

9. An apparatus for use in performing an electrophysiological test, comprising:
a bundle comprising a plurality of wires adapted for use in performing the physiological test, said plurality of wires being removably attached to each other.

10. The apparatus of claim 9, wherein the wires are attached to each other by an adhesive.

11. The apparatus of claim 9, further including a retainer for retaining the wires in the bundle.

12. The apparatus of claim 11, wherein the retainer comprises a ring adapted to move along the bundle in a longitudinal direction.

13. The apparatus of claim 9, further including a plug at one end of the bundle for interconnecting the plurality of wires.

14. An apparatus for use in performing an electrophysiological test, comprising:
an electrode including a recess;
a substance removably positioned in the recess; and
a removable cover for covering the conductive substance in the recess.

15. The apparatus of claim 14, further including a wire connected to the electrode.

16. The apparatus of claim 14, wherein the wire comprises a first portion detachably connected to a second portion including the electrode.

17. The apparatus of claim 16, wherein the first portion includes a connector for connecting with a device for receiving output signals from the wire in the course of the electrophysiological test.

18. An electrophysiological testing wire comprising a first segment removably attached to a second segment including an electrode.

19. The wire of claim 18, further including a connector for removably connecting the first and second segments.

20. The wire of claim 18, wherein the second segment is for single-use only.

21. A method for performing a medical test, comprising:
performing the medical test at a patient location;
displaying the results of the test at the patient location; and
transmitting the results of the test to a remote location.

22. The method of claim 21, wherein the displaying and transmitting steps occur simultaneously.

23. The method of claim 21, wherein the transmitting step comprises providing an input to a display device for performing the displaying step.
